(19) United States
(12) Patent Application Publication
(10) Pub. No.: US 2010/0030101 A1
(43) Pub. Date: Feb. 4, 2010

(54) Method And System For Acquiring Loudness Level Information

(76) Inventors: John D. Durrant, Allison Park, PA (US); Sheila Pratt, Pittsburgh, PA (US)

Correspondence Address: BAKER BOTTS L.L.P., 30 ROCKEFELLER PLAZA, 44TH FLOOR, NEW YORK, NY, 10112-4498, US

(21) Appl. No.: 12/479,476

(22) Filed: Jun. 5, 2009

Related U.S. Application Data
(60) Provisional application No. 61/059,602, filed on Jun. 6, 2008.

(51) Int. Cl. A61B 5/00

(52) U.S. Cl. 600/559

(57) ABSTRACT

The present invention relates to methods for acquiring hearing threshold values in a subject comprising providing a sound to the subject; providing a monitor for monitoring a movement response by the subject; monitoring the movement response by the subject to the sound; and progressively reducing the volume of the sound until no movement response by the subject to the sound occurs. The present invention further relates to methods and systems for acquiring loudness level information for a subject comprising providing a sound to a subject having a sensation level; providing a first sound to the subject having a sensational level; providing a monitor for monitoring a movement response by the subject; monitoring the movement response by the subject to the sound; and determining an elapsed reaction time between the sound and the movement response by the subject to the sound.

### CROSS-REFERENCE TO RELATED APPLICATION

This application claims priority to U.S. Provisional Patent Application Ser. No. 61/059,602, filed Jun. 6, 2008, the contents of which are hereby incorporated in its entirety herein.

### GRANT INFORMATION

Not applicable.

### 1. INTRODUCTION

The present invention relates to methods for acquiring hearing threshold values and methods and systems for acquiring loudness level information.

### 2. BACKGROUND OF THE INVENTION

The practice of pediatric audiology includes the hearing assessment of children for the diagnosis and treatment of hearing disorders. The target population is nominally children of age 6-18 months. Hearing analysis at this age is critical for determining speech language development, which is strongly dependent upon normal hearing function. Such hearing assessment is essential to fully characterize the degree and potential impact of any hearing loss. For example, hearing loss could occur as a result of congenital and early childhood disease (e.g., meningitis). Accordingly, such information is critical to providing optimum treatment and/or confirmation of successful treatment of hearing impairment with prosthetic devices. In order for such devices to successfully assist pediatric subjects, the devices should provide hearing assistance with the proper sensitivity levels appropriate for the degree of hearing impairment. In addition to determining sensitivity, optimal treatment requires knowledge of the subject's loudness precepts in order to strike an appropriate compromise between adequate sound audibility (especially speech sounds) and loudness comfort. This compromise is important to subject's ultimate acceptance of the hearing aid device.

Although hearing assessment and appropriate assistance is important at this early age, it is also an age in which children are unable to participate in conventional hearing testing because they lack the capacity to comprehend and follow the instructions of clinicians necessary to participate in such testing. Although non-behavioral, physiological methods are available to estimate hearing sensitivity in such difficult-to-test subjects (when compared with older children and adults), behavioral hearing confirmation and measurement remains the “gold standard” for clinical and research applications alike. The non-behavioral methods suffer from substantial drawbacks, such as the need for a sedated subject (natural sleep or drug-induced) and the uncertainty of ascertaining whether the physiological reaction to the sound is truly indicative that the child actually hears the sound.

Visual Reinforcement Audiometry (“VRA”) is a well developed behavioral hearing test that enables reasonably reliable hearing thresholds to be obtained from children who are too young to respond to conventional testing. VRA is effectively a “game” between the examiner and the subject in which the subject must be willing to participate, or “play.” Older children can be readily engaged in the same game as adults (e.g., raise hand or push-button when a tone or stimulus is heard) or in a more entertaining version (e.g., putting a toy in a bottle as a response or playing a computer game.) Very young children lack the capacity to play such games but can be conditioned to orient towards the source of the test sound, given appropriate reinforcement (e.g., animated toy becoming activated). Nozza (Ear and Hearing (1999), 20(6); 483-494) demonstrated that (at 2 kHz) VRA thresholds of infants from 6 to 9 months of age fall within 5.5 dB of normally hearing adults. Conventional methods in adults are considered to have 5-dB accuracy. Thus, when properly carried out, VRA can provide reasonably accurate and reliable audiometric thresholds for infants from 5 to 18 months.

However, VRA can be an inefficient method for many infants with hearing, cognitive and motor impairments, sometimes requiring multiple visits to obtain adequate information which, in turn, is never as comprehensive as routine audiometry in older children and adults. Thereafter, practical estimates of loudness have escaped clinicians for an even broader age range. While non-behavioral or “objective” measure exist (e.g., electrophysiological tests), these methods suffer other drawbacks, such as the need for a sedated patient (natural sleep or drug-induced) and the inevitable limitation of knowing if the child literally hears.

The inefficiency of VRA for threshold evaluation is also attributable to inherent procedural problems. For example, one is the variability of the behavior involved and the lack of salience associated with the stimuli needed to assess hearing. Infants vary in their responsiveness to sound, and habituate quickly to the tonal and narrowband noise stimuli often used in testing. Interesting sounds are likely to elicit a more obvious behavioral response that, in turn, will be more easily recognized by the tester. Less interesting sound stimuli may lead to more inconsistent, smaller and/or slower responses that may be missed.

Another problem is the child's possible reactions to the reinforcement. Reinforcement is a necessary component of VRA and is used to gain the child's attention and to develop a conditioned response to the sound stimuli. During VRA the infant is positioned on a parent's lap facing forward between at least two visual reinforcers placed at 45 and 315 degrees azimuth, or just to one side at 45 degrees azimuth. When testing in the sound field the loudspeakers are located near the reinforcers. Sounds first are presented at a suprathreshold level of sufficiently intense to elicit a clear orienting (head turn) response toward the sound source from the infant which, in turn, is associated with a visual reinforcer. The reinforcers typically consist of an illuminated animated toy or an animated figure on a video display. Once conditioned, the sound can be decreased in search of the threshold level. In this procedure, one audiologist or a computer runs the protocol and often another audiologist sits facing the infant, mildly distracting the infant in order to keep him/her at midline during silent and control intervals, and to reduce the number of false positive responses. Immediately after stimulus presentation (within 4 seconds for most babies) a headturn in the direction of the reinforcer is judged to be a response. However, infants do not always respond immediately, especially near threshold. Some infants with motor and cognitive impairments exhibit slowed responses, and many infants with hearing loss exhibit difficulty with sound localization when multiple loudspeakers are used. As a result, much rests on the ability of the examiner to judge a true response. Both efficiency and validity of the test, consequently, is vulnerable to compromise.

Infants not only habituate to the stimuli but habituation to the reinforcer also is a limitation. Once the reinforcement is no longer of interest infants stop responding and do not substantively recover even after a break. Although response efficiency may be increased through various manipulations of the basic VRA paradigm (Thompson, et al., Ear and Hearing (1992), 21(5); 471-487), there is only so much “work” one can expect from these very young subjects. Data collection is clearly constrained much more than in older children and adults, regardless of the conditioning paradigm, and novelty and variability of the reinforcement. This suggests a need to improve response observation and measurement.

For infants who fail to demonstrate clear head turns (e.g., overt responses to the stimuli) during VRA and who tend to demonstrate a fair amount of “ambient” head movement, it may be necessary to discern smaller, less obvious, behaviors, the conventional VRA approach may not be effective in identifying responses.

### 3. SUMMARY OF THE INVENTION

The present invention relates to a method for acquiring hearing threshold values in a subject, which method comprises providing a sound to the subject, providing a monitor for monitoring a movement response by the subject, monitoring a movement response by the subject to the sound, and progressively reducing the volume of the sound until no movement response by the subject to the sound occurs.

In some embodiments, monitoring the movement response by the subject to the sound comprises monitoring head turn response of the subject. In some embodiments, monitoring the movement response by the subject to the sound comprises monitoring eye response of the subject.

In some embodiments, providing a monitor comprises providing a mechanical rate monitor to the subject. In some embodiments, providing a monitor comprises providing a video image monitor to the subject. In some embodiments, the video image monitor comprises an imaging device for providing a video image of the subject and a processor running software adapted to detect the movement response of the subject by evaluating the video image of the subject. In some embodiments, providing a monitor comprises providing an electrophysiological recording instrument. In some embodiments, the electrophysiological recording instrument is an electromyograph. In some embodiments, the electrophysiological recording instrument is an electrooculograph.

The present invention further relates to a method for acquiring loudness level information for a subject, which method comprises providing a sound to a subject having a sensation level; providing a monitor for monitoring a movement response by the subject; monitoring the movement response by the subject to the sound; and determining an elapsed reaction time between the sound and the movement response by the subject to the sound.

In some embodiments, the method further comprises correlating the elapsed reaction time to the sensation level of the first sound. In some embodiments, the method further comprises a second sound to the subject having a sensation level, monitoring the movement response by the subject to the second sound, determining an elapsed reaction time between the second sound and the movement response by the subject to the second sound, correlating the elapsed reaction time to the sensation level of the second sound, and determining a rate of change between the elapsed reaction time to the first sound and the elapsed reaction time to the second sound. In some embodiment, the method further comprises determining a loudness parameter based on the lapsed reaction time.

In some embodiments, monitoring the movement response by the subject to the first and/or second sound comprises monitoring head turn response of the subject. In some embodiments, monitoring the movement response by the subject to the first and/or second sound comprises monitoring eye response of the subject.

In some embodiments, providing the monitor comprises providing a mechanical rate monitor to the subject. In some embodiments, providing the monitor comprises providing a video image monitor to the subject. In some embodiment, the video image monitor comprises an imaging device for providing a video image of the subject and a processor running software adapted to detect the movement response of the subject by evaluating the video image of the subject. In some embodiments, providing a monitor comprises providing an electrophysiological recording instrument. In some embodiments, the electrophysiological recording instrument is an electromyograph. In some embodiments, the electrophysiological recording instrument is an electrooculograph.

The present invention further relates to a system for acquiring hearing threshold values in a subject, which system comprise one or more loudspeakers adapted to provide sounds having progressively reduced volume, and a monitor associated with the subject to track movement response by the subject to the sounds.

In some embodiments, the monitor comprises a mechanical rate sensor mounted on the subject to track movement of the subject. In some embodiments, the monitor comprises an imaging device for providing a video image of the subject and a process running software adapted to detect the movement response of the subject by evaluating the video image of the subject. In some embodiments, the monitor comprises an electrophysiological recording instrument. In some embodiments, the electrophysiological recording instrument is an electromyograph. In some embodiments, the electrophysiological recording instrument is electrooculograph.

In some embodiments, the movement response by the subject to the sounds is head turn response of the subject. In some embodiments, the movement response by the subject to the sounds is eye response of the subject.

### 4. BRIEF DESCRIPTION OF THE FIGURES

FIG. 1. User interface displaying a time plot useful for a calibration procedure wherein the subject turns their head to track a small dot from center to 10 degrees to the right and 10 degrees to the left.

FIG. 2. User interface displaying a video playback window of the head of the subject and time plots illustrating movement of the subject's head.

FIG. 3. User interface displaying time plots of movement of the subject, e.g., position as measured by video tracking, computed head velocity, and measured head velocity.

FIG. 4A. Plot of responses of subjects as a function of sensation level comparing head turn vs. push-button response.

FIG. 4B. Plot of percentage of responses of subjects as a function of sensation level, comparing head turn vs. push-button responses.

FIG. 5. Plot of percentage of responses across all subjects, excluding incorrect head turns.

FIG. 6. Plot of reaction times across all subjects as a function of sound level.

FIG. 7. Schematic view of a system in accordance with an exemplary embodiment of the disclosed subject matter.

### 5. DETAILED DESCRIPTION OF THE INVENTION

The system and methods described herein are useful for acquiring loudness related information for a subject. Although the description herein concerns the example of hearing assessment of adult subjects, the system and methods herein are believed useful for other subjects such as infants and young children.

The subject matter includes two components: (1) an improved response measurement protocol for VRA (e.g., estimation of hearing thresholds in infants) and reaction time measurements, and (2) a loudness-related metric.

The head turn response of VRA is measured mechanically for machine assisted scoring and computational analysis, which overcomes the limitations of the examiner relying on subjective scoring via visual inspection. The head-turn may be measured using an angular rate sensor, as used in head-only rotation in vestibular testing. Alternatively, video monitoring permits testing without physical encumbrance of the subject. Velocity of head turn is measured directly (using mechanical rate sensor) or computed from head displacement (via video tracking), which provides a sharp definition of response occurrence. The reaction time provides information to characterize responses near the threshold, which can enhance the interpretation of the nature and quality of the subject's behavior around the limit of hearing (threshold).

Head and eye movement may also be measured by relying on, e.g., electrophysiological recording instruments, such as electro-oculograph and electromyograph. An “electrophysiological recording instrument” used herein, is an instrument which relies on electrophysiology or electrophysiological techniques. Electrophysiology is the study of the electrical properties of biological cells and tissues. It involves measurements of voltage change or electric current on a wide variety of scales from single ion channel proteins to whole organs like the heart. Classical electrophysiology techniques involve placing electrodes into various preparations of biological tissue. The principal types of electrodes are: 1) simple solid conductors, such as discs and needles (singles or arrays), 2) tracings on printed circuit boards, and 3) hollow tubes filled with an electrolyte, such as glass pipettes. The principal preparations include 1) living organisms, 2) excised tissue (acute or cultured), 3) dissociated cells from excised tissue (acute or cultured), 4) artificially grown cells or tissues, or 5) hybrids of the above. Many particular electrophysiological readings/recordings have specific names, for example, “electrocardiography” is for the heart, “electroencephalography” is for the brain, “electrocorticography” is for the cerebral cortex, “electromyography” is for the muscles, “electrooculography” is for the eyes, “electroretinography” is for the retina, and “electroantennography” is for the olfactory receptors in arthropods.

An “electrooculograph” used herein, is an instrument which performs electromyography (EOG). An electrooculograph is an instrument which records eye movement by measuring small electrical charges with tiny electrodes attached to the inner and outer corners of the eye. Electrooculography is a technique for measuring the resting potential of the retina. The resulting signal is called the electrooculogram. The main applications are in opthalmological diagnosis and in recording eye movements. Unlike the electroretinogram, the EOG does not represent the response to individual visual stimuli. Usually, pairs of electrodes are placed either above and below the eye or to the left and right of the eye. If the eye is moved from the center position towards one electrode, this electrode “sees” the positive side of the retina and the opposite electrode “sees” the negative side of the retina. Consequently, a potential difference occurs between the electrodes. Assuming that the resting potential is constant, the recorded potential is a measure for the eye position.

An “electromyograph” used herein, is an instrument which performs electromyography (EMG). Electromyography is a technique for evaluating and recording the activation signal of muscles. EMG is performed using an instrument called an electromyograph, to produce a record called an electromyogram. An electromyograph detects the electrical potential generated by muscle cells when these cells are mechanically active, and also when the cells are at rest. The signals can be analyzed in order to detect medical abnormalities or analyze the biomechanics of human or animal movement.

The electrophysiology techniques including electrooculography (EOG) and electromyography (EMG) and its applications are well known in the art.

Using the same instrumentation with suprathreshold stimulation, head turn reaction time is measured as a function of stimulus level. Obtaining the change in reaction time as a function of time permits for the gathering of statistics such as the slope of the function. Moreover, reaction time provides a metric by which to judge perceived loudness. Loudness, per se, can be estimated by combining average reaction time values with published loudness data, e.g., Steven's loudness scale, obtained in normally hearing subjects using direct magnitude estimation. The loudness scale, for example, predicts that a 10 dB change in sound level is needed (overall) to double the sensation of loudness. The subject's reaction time profile can be matched to the average profile and shifted along the sound level axis for best fit. Loudnesses, measured in sones, for each sound level of interest can simply be read off from intersections with the loudness (“power”) function.

Small head turns or “noisy” head movement can be scored with the assistance of sensitive and consistent measurements of head-turn responses acquired with the aid of computer sampling.

One measure determined by the method and system described herein was audiometric threshold (limit of sound detection), a comparison between results obtained through conventional test methods and a VRA-like procedure using head orientation responses evaluated with the aid of computer video tracking and head mechanical motion systems. The two types of head-turn measurement were used for cross-validation. Additionally, the relationship between reaction time of head motion and reaction time of button pressing in response to both near-threshold and supra-threshold sound stimuli were examined.

The responses of one or more subjects were evaluated. For example, a sample of 10 adult subjects was examined. In this exemplary case, the subjects were graduate students who had normal hearing sensitivity by conventional audiometry, and had negative otologic and neurotologic histories. In this exemplary case, female subjects were tested due to broad availability as females predominate the student body of the principle investigator's department from which the subjects were recruited. Hearing within normal limits was defined, according to conventional clinical standards as thresholds no greater than 25 dB HL at 0.25, 1, 2, 4, or 8 kHz.

The experimental protocol consisted of study components, each comprising two paradigms (push-button response and head turn response). The two components addressed, respectively, issues of threshold search and loudness-reaction time scaling.

### Threshold Analysis

The subject's pure tone threshold for a 2 kHz warble tone was obtained in the sound field (i.e. through loudspeakers in a sound treated booth); the “warble” tone is a lightly frequency-modulated tone used to discourage standing-wave formation the test suite. A preliminary determination was made using a method close to the clinical procedure, but permitting threshold resolution within 1 dB. Specifically, the up-down or staircase method (an adaptation of the classical method of limits) was used with 10 reversals, but ignoring the first two ascents/descents and reversals for the subject to settle into the vicinity of their putative threshold. The average level of the last 8 reversals was taken as the threshold and reference level for the main threshold and loudness components.

A comparison of auditory thresholds obtained through conventional methods using a classical push-button response and head turn response as used in conventional VRA, facilitated by objective head motion tracking was performed. The classical psychophysical method of constant stimuli (MCS) was utilized for threshold data acquisition, that is, thresholds to be analyzed and compared between conditions. This method yields a full psychometric function directly. Expected are response rates as a function of stimulus level that follow overall the “ogive” function (cumulative normal distribution), representing the systematic growth and saturation at/near 100% response at the highest stimulus level, thereby permitting the observation of subtle differences in growth of percent response and/or its variance. The MCS requires sets of tones varying in intensity. For the push-button response audiometry, a range of 20 dB was used, centered on the putative level derived from up-down tracking (above) and using a total of 11 2-dB steps. Each stimulus was repeated 10 times per step, randomized over steps and repetition. The subject faced straight ahead and was asked to press a hand-held response button as soon as the presence of sound was detected. Per classical principles, threshold was evaluated as the intensity level at which the stimulus was detected 50% of the time that it was presented.

The paradigm used in the VRA-like (head-turn response) audiometry was substantially identical to protocol as above for push-button response, except that the subject was instructed to respond by turning her head toward the apparent origin of the sound. The sound could occur randomly to the right or left with the loudspeakers situated at ±45 degrees azimuth. This variant was employed in deference to the likelihood that these adult subjects could quickly learn to anticipate the stimulus if the sound persistently comes from just one side and that adults (unlike infants) are not rewarded substantively by seeing an amusing toy over the loudspeaker of origin (per the principle of VRA, per se).

Video imaging was obtained with an imaging device, such as a digital camera, interfaced to a personal computer wherein the image could be analyzed by using a process running software designed to track a high-contrast moving feature, as is known in the art.

Alternatively, head response information could be obtained from a mechanical rate sensor worn on the head, e.g., mounted on a hard-hat suspension frame. This head ware is light and comfortable while providing a stable mount for the mechanical rate sensor, as it is adjustable to fit snuggly on the individual subject's head. Combined comfort and stability was assured by foam padding along the head-band. As the rate of human head turns are relatively low velocity, tight coupling was not required for purposes of this study, avoiding direct skin contact with the transducer itself and use of adhesives or an extremely tight-fitting headwear.

In this exemplary configuration, a transduction element used was a piezoelectric transducer (Watson rate sensor), which has been used widely in vestibular research. The helmet band also served to hold a small target (black circle) on cloth background (white) for purposes of video tracking. This accomplished two objectives—(1) obscuring the subject's visual fixation on the loudspeakers and (2) permitting use of high-resolution software for tracking movement. For example, the software can tack saccadic movements of the pupils, i.e., in the testing of nystagmus. As the reset phase of the eye saccade is fairly high velocity, more than adequate tracking of head turns, including precise quantification of head-turn reaction time could be calculated by this technique.

Video imaging obtained from the digital camera was cross-validated with the signal from the mechanical rate sensor. The mechanical rate sensor readily matched the performance of the video imaging, demonstrating excellent sensitivity to head-turn characteristics found with subjects orienting to the origin of the test signal.

### Loudness-Related Reaction Time

The loudness-related reaction time analysis described herein involved the comparison of button-pushing and head-turn reaction times to a set of suprathreshold stimuli administered via MCS. In these exemplary cases, the set of 2 kHz tones spanned 20 to 80 dB SL. The “SL” refers to sensation level, which is the decibel sound pressure level above the threshold of detection. To cover this range, steps were 4 dB, stimulus level was randomized across steps and repetitions (10 per step for a total of 11 steps), and SL was referenced to the putative threshold obtained using the staircase method (see above). It has long been known that there is an inverse relationship between auditory stimulus level and reaction time (e.g. Chocholle, Annee Psychologique (1940), 41, 65-124; therefore, it was expected that with increasing SL, reaction time was decreased systematically.

Button-pushing reaction time was measured using Adobe® Audition™ software by comparing the digital recordings of the test stimuli, a marker for the button press, and the head-turn signal. Audition™ software permits fine measurements of multi-track signals to permit open-ended approaches to signal conditioning as needed (e.g. filtering), and yet have a fairly precise (if tedious) numerical analysis capability. As before, head-turn reaction time was indexed in two ways: (a) video-tracked and (b) mechanically tracked head-turn. The mechanical rate sensor, incidentally, is largely responsive only to horizontal motion. As the desired and most relevant head turn behavior is evoked in the context of horizontal binaural sound localization, by definition in the horizontal plane, this indeed was a useful cross-validation method. As discussed above, right and left head orientations was elicited randomly, and all testing occurred in a sound treated room with loudspeakers situated to the right and left of the subject.

### Efficacy of Head-Turn Tracking

The video tracking system, e.g., the adapted eye-tracking system, proved to be efficacious for purposes of head-turn tracking. Both video and mechanically tracked head-turn velocity are useful to determine reaction time, name the time between the stimulus onset (not illustrated) and first significant deviation from the average baseline.

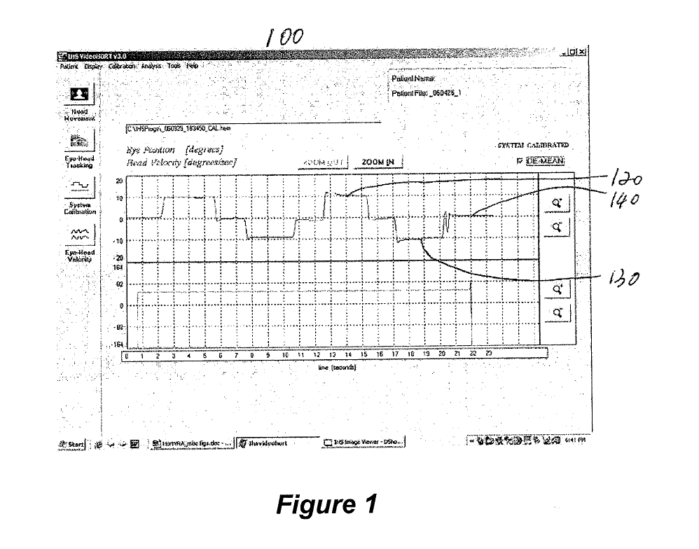

FIG. 1 represents a user interface 100 for the calibration procedure wherein the subjects were required to turn their head to track a small dot from center to 10 degrees to the right (position 120) and 10 degrees to the left (position 130). This tracking data is represented by the upper line 140 in FIG. 1.

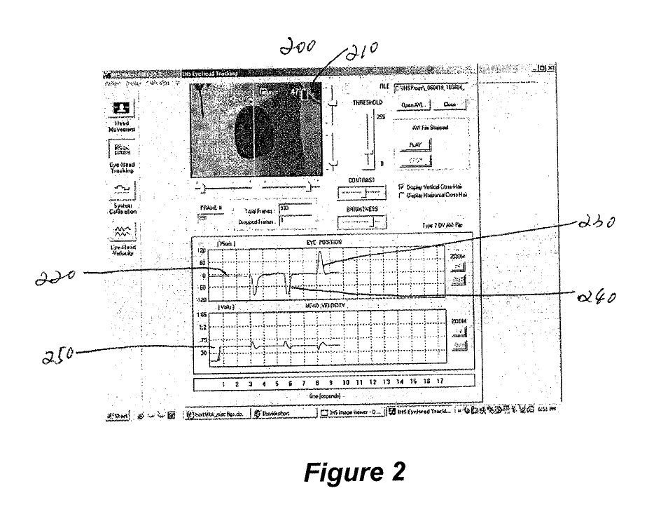

FIG. 2 represents the user interface 200 for the analysis of the head velocity. The black target appears in the video playback window 210, showing that the entire target is being properly identified by the computer-image processing. Below are tracings showing the successful video tracking of head turns. The upper tracing 220 shows the angular displacement of the target (following the head turn) with upward deflection 230 representing a turn to the right and downward deflection 240 a turn toward the left. The lower tracing 250 indicates the head turn velocity as measured by the rate sensor. It can be observed that the tracings line up quite well, although in opposite phase. In another embodiment, the phases may be reversed.

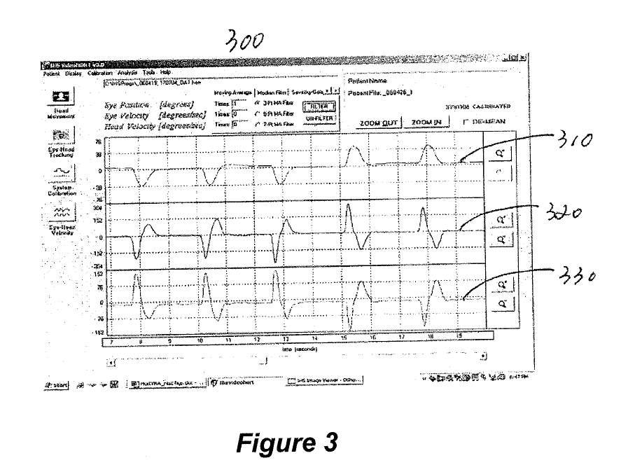

FIG. 3 illustrates a user interface 300 illustrating the comparison of head position as measured by video tracking (top tracing 310), head velocity computed from video computation (middle tracing 320), and head velocity as measured by the mechanical rate sensor (bottom tracing 330). The opposition between phase of the video and mechanical measurements illustrated in the exemplary embodiment may be modified in additional embodiments. It is evident from comparison of video tracking and rate sensor data that both techniques are acceptably accurate.

### 6. EXAMPLE

The results of this preliminary study have been evaluated much as would be more conventional threshold data obtained using classical psychophysical methods. It is well established that such methods suffer from a lack of complete control of subject criterion, making the results vulnerable to subject response and other biases. Forced-choice paradigms, based on the signal detection theory, offer solutions to such problems but were not relied upon herein. On the other hand, the effects of response bias on the psychometric function are also well known/predictable (e.g., see Gescheider, Psychophysics: The Fundamentals, 3^{rd} edition (1997)). Namely, should the subject lower his/her criterion for response (i.e., more liberal), the entire ogive naturally is shifted to the left, and vice versa if his/her criterion is raised (i.e., more conservative). In the exemplary embodiment, it was presumed that reaction time can be interpreted correspondingly, and the subjects' data was separated accordingly into two groups-lower-than-average reaction time (faster) and higher-than-average (slower) reaction times, corresponding to liberal versus conservative responses, respectively.

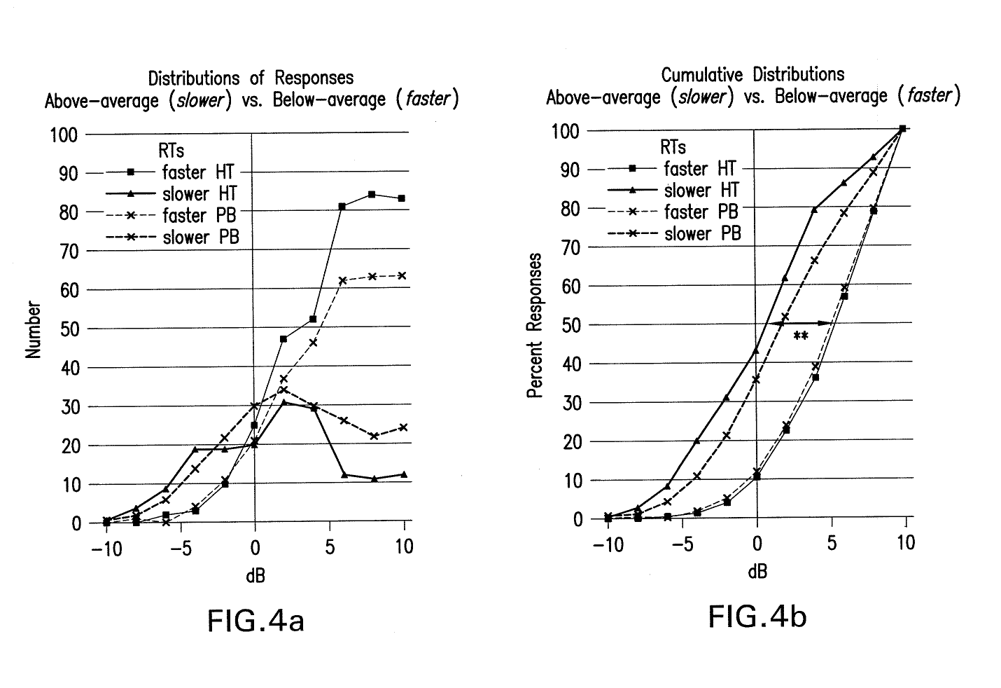

FIG. 4a illustrates the number of head turn responses that occur with varying sensation level (SL). For the “faster” head turn response and “faster” push-button response, the number of responses increased as the SL of the sound increased. In the case of the “slower” head turn response and “slower” push-button response, the number of responses increased with increasing SL, peaked, and then decreased with increasing SL. Although not completely understood, it is believed that as the signal exceeds 0 dB SL, the listeners have more certainty (less ambiguity) about whether the test signal was present. With increased certainty more of the subjects' responses were considered faster. Proportion-of-response functions (FIG. 4a) were transformed to provide the results in FIG. 4b and reflect the anticipated shift in the cumulative distributions. The percentage of each category of response (type and speed) was calculated in a cumulative fashion from −10 dB SL to +10 dB SL, so that at −10 dB SL the percentage points on the functions relate only to the percentage of responses at −10 dB SL, but at +10 dB SL the percentage points on the function represent the accumulation of the percentages from each SL from −10 through +10 dB. The range of stimulus levels used did not permit robust characterization of asymptotes, but did provide for robust characterization of the critical sloped portion of the function, i.e. the segment that captures threshold. These results (means) show very little difference between push-button and head-turn results. It may seem counter-intuitive that there should be such comparability, since the head-turn task evokes choice, rather than simple, reaction times. Although actual comparisons of reaction times themselves (not shown) were not entirely overlapping between modes, they were largely overlapping between test modes near and above threshold. The explanation is believed to be found in the natural efficiency of the head-turn reflex-like reactions of orienting behavior. While not needed to localize sounds, head turn response to seek the sound is quite a natural behavior for sounds of special interest or that are startling.

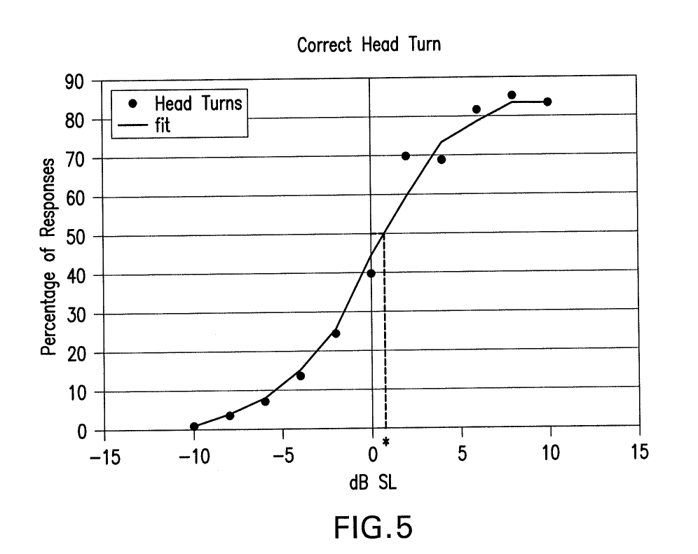

In FIG. 5, the head-turn results are provided across all subjects, regardless of overall reaction time, but excluding incorrect head turns (i.e., erroneous choice of the right versus left loudspeaker). For example, after removing all responses in which errors of choice were observed (i.e., right vs. left), the percentage of correct detection of the test stimuli was calculated as a function of SL. This actually proved to make little difference in the threshold estimate, which in turn was within a decibel of the average thresholds predetermined by the staircase method (and known well to provide comparably accurate estimates of threshold). The asterisk serves to highlight the estimated threshold level as defined by 50% correct on the performance-intensity function. Dispersion of the data is approximately 5 dB, which corresponds well to clinical threshold variability. It thus was concluded that, at least in adults, head-turn reaction time measures overlap simple reaction time results via push-button response and are fully characteristic of classical psychometric responses near sensory thresholds. As reaction time actually is more than a mere yes-no response, this method provides greater information, potentially interpretable with respect to stability of the subject's criterion of response, and thus a broader information base by which to evaluate subjects' responses. Such information could provide not only the desired threshold estimates but also robust quantitative assessments of quality of the results.

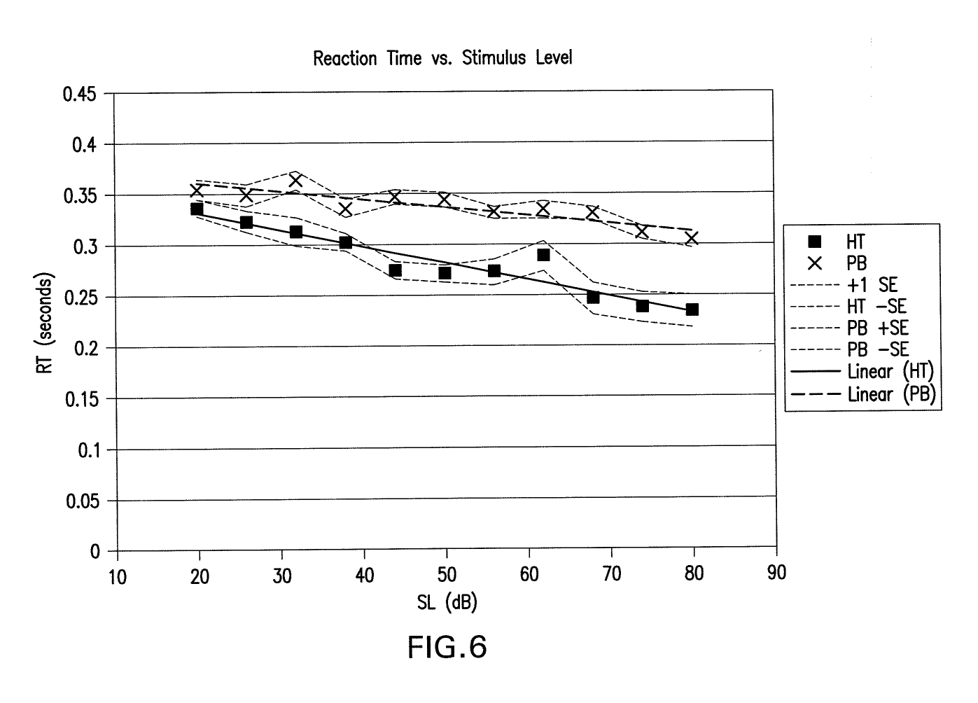

The mean reaction times obtained with increasing levels of supra-threshold stimuli also demonstrated comparable results between the two test modalities overall. Although not showing a gain in reaction time across all subjects, as suggested in FIG. 6, results for the majority of subjects, and thus the mean reaction times, predict equal or shorter reaction times for head-turn than push-button responses. Both sets of data demonstrate shorter reaction times with increasing sound level, as expected from the literature. On the other hand, consistent across subjects was the tendency for head-turn reaction times to show a greater slope as a function of stimulus level. It is believed that this is a particularly important finding in its own right, as it makes it more likely that (at least) broad categories—soft, medium or comfortable, and loud—may be accurately estimated from such reaction time measures. Namely, with a steeper slope, statistical significance of changes in reaction time over level is more likely to be realized, specifically in the single-subject design of clinical tests. The statistical significance is suggested by the ±standard error (SE) intervals outlined by the dotted graphs.

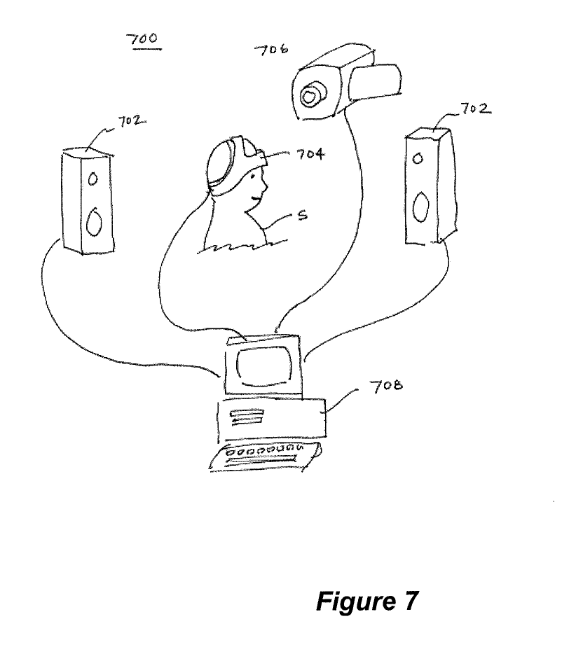

An exemplary system for carrying out the methods for acquiring hearing threshold values is discussed herein, and further illustrated in FIG. 7. The system 700 may include one or more loudspeakers 702, or other devices for providing sound to the subject. Headphones may be used in certain embodiments. The loudspeakers 702 provide sounds which may be progressively reduced in volume. A monitor 704 is associated with the subject S to track movement responses by the subject S to the sounds. The monitor 704 may include a mechanical rate sensor mounted on the subject to track movement of the subject. The system 700 may also include an imaging device 706, such as a digital camera, which provides a video image of the subject. A computer 708, such as a personal computer including a processor, is provided which runs software adapted to detect the movement response of the subject by evaluating the video image of the subject as discussed herein.

It will be understood that the foregoing is only illustrative of the principles of the disclosed subject matter, and that various modifications can be made by those skilled in the art without departing from the scope and spirit thereof. For example, although the above head-turn reaction time measures have been performed with cooperative young adults, it is believed that such technique is applicable equally in infants and young children. It is further believed that via the use of eye-tracking technology, wherein eye, not just head, turns can be tracked, the techniques described herein are applicable to the age range about 3-6 months.

What is claimed is:

1. A method for acquiring hearing threshold values in a subject comprising:
providing a sound to the subject;
providing a monitor for monitoring a movement response by the subject;
monitoring the movement response by the subject to the sound; and
progressively reducing the volume of the sound until no movement response by the subject to the sound occurs.

2. The method of claim 1, wherein monitoring the movement response by the subject to the sound comprises monitoring head turn response of the subject.

3. The method of claim 1, wherein monitoring the movement response by the subject to the sound comprises monitoring eye response of the subject.

4. The method of claim 1, wherein providing the monitor comprises providing a mechanical rate monitor to the subject.

5. The method of claim 1, wherein providing the monitor comprises providing a video image monitor to the subject.

6. The method of claim 5, wherein the video image monitor comprises:
an imaging device for providing a video image of the subject; and
a processor running software adapted to detect the movement response of the subject by evaluating the video image of the subject.

7. The method of claim 1, wherein providing the monitor comprises providing an electrophysiological recording instrument.

8. The method of claim 7, wherein the electrophysiological recording instrument is an electromyograph.

9. The method of claim 7, wherein the electrophysiological recording instrument is an electrooculograph.

10. A method for acquiring loudness level information for a subject comprising:
providing a first sound to the subject having a sensation level;
providing a monitor for monitoring a movement response by the subject;
monitoring the movement response by the subject to the first sound; and
determining an elapsed reaction time between the first sound and the movement response by the subject to the first sound.

11. The method of claim 10, further comprising correlating the elapsed reaction time to the sensation level of the first sound.

12. The method of claim 11, further comprising providing a second sound to the subject having a sensation level, monitoring the movement response by the subject to the second sound, determining an elapsed reaction time between the second sound and the movement response by the subject to the second sound, correlating the elapsed reaction time to the sensation level of the second sound, and determining a rate of change between the elapsed reaction time to the first sound and the elapsed reaction time to the second sound.

13. The method of claim 10, further comprising determining a loudness parameter based on the elapsed reaction time.

14. The method of claim 10, wherein monitoring the movement response by the subject to the first sound comprises monitoring head turn response of the subject.

15. The method of claim 12, wherein monitoring the movement response by the subject to the second sound comprises monitoring head turn response of the subject.

16. The method of claim 10, wherein monitoring the movement response by the subject to the first sound comprises monitoring eye response of the subject.

17. The method of claim 12, wherein monitoring the movement response by the subject to the second sound comprises monitoring eye response of the subject.

18. The method of claim 10, wherein providing the monitor comprises providing a mechanical rate monitor to the subject.

19. The method of claim 10, wherein providing the monitor comprises providing a video image monitor to the subject.

20. The method of claim 19, wherein the video image monitor comprises:
an imaging device for providing a video image of the subject; and
a processor running software adapted to detect the movement response of the subject by evaluating the video image of the subject.

21. The method of claim 10, wherein providing the monitor comprises providing an electrophysiological recording instrument.

22. The method of claim 21, wherein the electrophysiological recording instrument is an electromyograph.

23. The method of claim 21, wherein the electrophysiological recording instrument is an electrooculograph.

24. A system for acquiring hearing threshold values in a subject comprising:
one or more loudspeakers adapted to provide sounds having progressively reduced volume; and
a monitor associated with the subject to track movement response by the subject to the sounds.

25. The system of claim 24, wherein the monitor comprises a mechanical rate sensor mounted on the subject to track movement of the subject.

26. The system of claim 24, wherein the monitor comprises:
an imaging device for providing a video image of the subject; and
a processor running software adapted to detect the movement response of the subject by evaluating the video image of the subject.

27. The system of claim 24, wherein the movement response by the subject to the sounds is head turn response of the subject.

28. The system of claim 24, wherein the movement response by the subject to the sounds is eye response of the subject.

29. The system of claim 24, wherein the monitor comprises an electrophysiological recording instrument.

30. The system of claim 29, wherein the electrophysiological recording instrument is an electromyograph.

31. The system of claim 29, wherein the electrophysiological recording instrument is an electrooculograph.
